(19) United States
(12) Patent Application Publication
(10) Pub. No.: US 2024/0077507 A1
(43) Pub. Date: Mar. 7, 2024

(54) SAMPLE ANALYSIS APPARATUS AND SAMPLE ANALYSIS METHOD USING THE SAME

(71) Applicant: SAMSUNG ELECTRONICS CO., LTD., Suwon-si (KR)

(72) Inventors: Seonhwa Go, Suwon-si (KR); Younghye Kim, Suwon-si (KR); Jiyun Lim, Suwon-si (KR); Sangyoon Shin, Suwon-si (KR)

(73) Assignee: SAMSUNG ELECTRONICS CO., LTD., Suwon-si (KR)

(21) Appl. No.: 18/234,692

(22) Filed: Aug. 16, 2023

(30) Foreign Application Priority Data
Sep. 6, 2022 (KR) 10-2022-0113051
Nov. 14, 2022 (KR) 10-2022-0151997

(51) Int. Cl. G01N 35/02; G01N 35/10

(52) U.S. Cl. CPC G01N 35/025; G01N 35/1009; G01N 2035/0465

(57) ABSTRACT

A sample analysis apparatus includes a first chamber that performs a first heating process for heating a small sample and a second chamber including a second chamber housing in which a second heating process for heating a wafer sample is performed. The first chamber includes a first chamber housing in which the first heating process is performed, a first support that supports the small sample, a first heating device that heats the small sample, an accommodation housing in which the small sample is accommodated, a sample tray arranged in the accommodation space, sample container configured to store the small sample, and a transfer module configured to transfer the sample container and the small sample from the accommodation housing to the first chamber.

### CROSS-REFERENCE TO RELATED APPLICATIONS

This application is based on and claims priority under 35 U.S.C. § 119 to Korean Patent Application No. 10-2022-0113051, filed on Sep. 6, 2022, and to Korean Patent Application No. 10-2022-0151997, filed on Nov. 14, 2022, in the Korean Intellectual Property Office, the disclosures of each of which being incorporated by reference herein in their entireties.

### BACKGROUND

The present disclosure relates to a sample analysis apparatus and a sample analysis method using the same and, more specifically, to a sample analysis apparatus including a mass spectrometer for analyzing the mass of a sample and a sample analysis method using the sample analysis apparatus.

With the development of the electronics industry, semiconductor devices have become more integrated and sophisticated. According to the trend of integration and advancement of the semiconductor devices, the influence of a reactive gas used in a process of manufacturing the semiconductor devices on quality of the semiconductor devices has gradually increased. Accordingly, qualitative and quantitative analysis of the reactive gas used in the process of manufacturing the semiconductor devices is required. In general, a standard sample including a reactive gas in a preset content is required to perform quantitative analysis of a reactive gas. However, there is a difficulty in simultaneously analyzing a standard sample having a shape of a silicon wafer and a standard sample having a shape other than the silicon wafer due to structural limitations of a reactive gas analysis apparatus including only one chamber, and a solution to address this matter is required.

### SUMMARY

It is an aspect to provide a sample analysis apparatus capable of effectively analyzing various types of samples and a sample analysis method using the sample analysis apparatus.

According to an aspect of one or more embodiments, a sample analysis apparatus comprising a first chamber configured to perform a first heating process for heating a small sample to generate a first reactive gas, the first chamber comprising a first chamber housing including a first processing space in which the first heating process is performed; a first support configured to support the small sample while the first heating process is performed; a first heating device arranged on a lower surface of the first support and configured to heat the small sample; an accommodation housing including an accommodation space in which at least one small sample is accommodated, the accommodation housing being connected to the first chamber housing; a sample tray arranged in the accommodation space; at least one sample container arranged on the sample tray and configured to store the at least one small sample; and a transfer module configured to transfer a sample container of the at least one sample container that stores the small sample from the accommodation space to the first processing space; and a second chamber including a second chamber housing including a second processing space in which a second heating process for heating a wafer sample to generate a second reaction gas is performed, a second support configured to support the wafer sample while the second heating process is performed, and a second heating device arranged on a lower surface of the second support and configured to heat the wafer sample.

According to another aspect of one or more embodiments, a sample analysis apparatus comprising a first chamber configured to perform a first heating process for heating a small sample to generate a first reactive gas, the first chamber comprising a first chamber housing including a first processing space in which the first heating process is performed; a first support configured to support the small sample while the first heating process is performed; a first heating device arranged on a lower surface of the first support and configured to heat the small sample; an accommodation housing including an accommodation space in which at least one small sample is accommodated, the accommodation housing being connected to the first chamber housing; a sample tray arranged in the accommodation space; at least one sample container arranged on the sample tray and configured to respectively store the at least one small sample; and a transfer module configured to transfer a sample container of the at least one sample container that stores the small sample from the accommodation space to the first processing space; a second chamber including a second chamber housing including a second processing space in which a second heating process for heating a wafer sample to generate a second reactive gas is performed, a second support configured to support the wafer sample while the second heating process is performed, and a second heating device arranged on a lower surface of the second support and configured to heat the wafer sample; an analysis device configured to analyze a physical property of the at least one small sample by using the first reactive gas and a physical property of the wafer sample by using the second reactive gas; a first pipe configured to move the first reactive gas from the first chamber toward the analysis device; a second pipe configured to move the second reactive gas from the second chamber toward the analysis device; and a third pipe connected to the first pipe and the second pipe and configured to move the first reactive gas moving in the first pipe and the second reactive gas moving in the second pipe toward the analysis device.

According to yet another aspect of one or more embodiments, a sample analysis method comprising opening a first chamber gate and an analysis device valve; transferring a sample container accommodated in an accommodation space in an accommodation housing and a small sample stored in the sample container into a first processing space in a first chamber housing by using a transfer module; performing a first heating process on the small sample; opening a first chamber valve to move a first reactive gas generated by the first heating process to an analysis device; closing the first chamber valve and the analysis device valve after analysis of the first reactive gas is completed; providing a wafer sample to a second processing space in a second chamber; performing a second heating process on the wafer sample; sequentially opening the analysis device valve and a second chamber valve to move a second reactive gas generated by the second heating process to the analysis device; and closing the second chamber valve and the analysis device valve after analysis of the second reactive gas is completed.

### BRIEF DESCRIPTION OF THE DRAWINGS

The above and other aspects will be more clearly understood from the following detailed description taken in conjunction with the accompanying drawings, in which:

FIG. 1 is a schematic configuration diagram of a sample analysis apparatus according to some embodiments;

FIG. 2A is an enlarged cross-sectional view of a portion POR of FIG. 1; FIG. 2B is a plan view of the portion POR of FIG. 1;

FIGS. 3A, 4A, 5A, and 6 are configuration diagrams illustrating operating methods of a sample analysis apparatus according to some embodiments;

FIGS. 3B, 4B, and 5B are enlarged cross-sectional views illustrating operating methods of a sample analysis apparatus according to some embodiments; and

FIGS. 3C, 4C, and 5C are plan views illustrating operating methods of a sample analysis apparatus according to some embodiments.

### DETAILED DESCRIPTION

Hereinafter, various embodiments will be described in detail with reference to the accompanying drawings. The same reference numerals are used for the same components in the drawings, and redundant descriptions thereof are omitted.

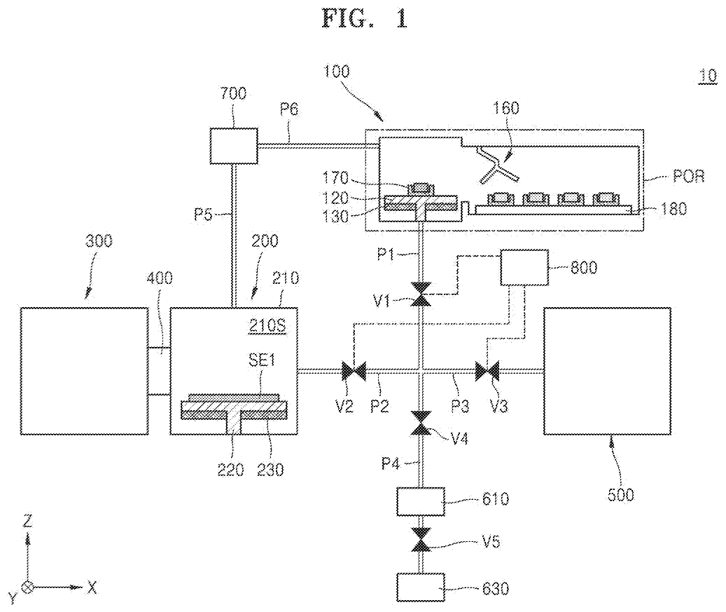

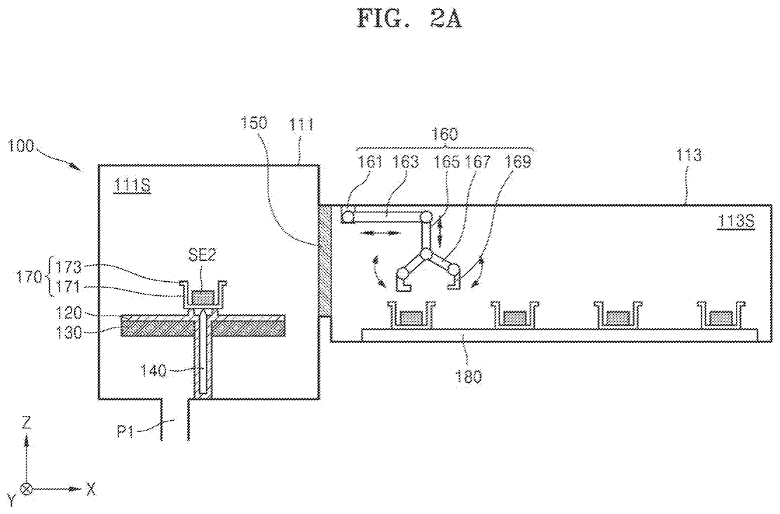

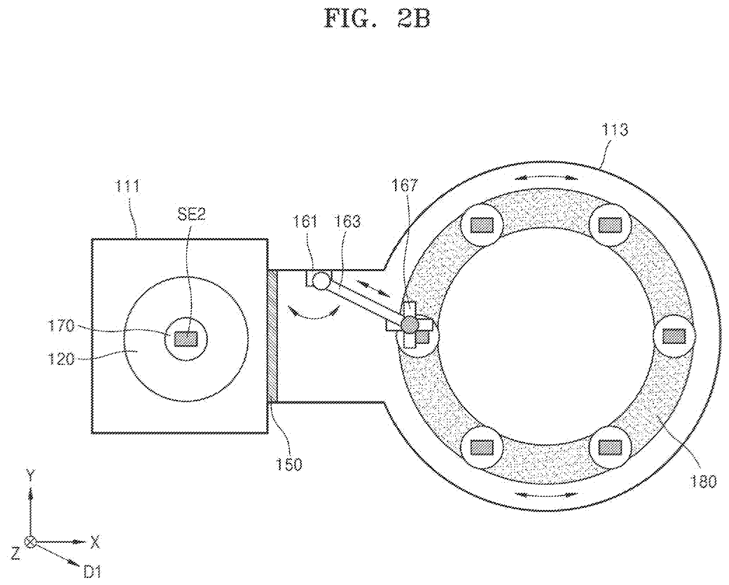

FIG. 1 is a schematic configuration diagram of a sample analysis apparatus 10 according to some embodiments. FIG. 2A is an enlarged cross-sectional view of a portion POR of FIG. 1. FIG. 2B is a plan view of the portion POR of FIG. 1.

Referring to FIGS. 1, 2A, and 2B, the sample analysis apparatus 10 may include a first chamber 100, a second chamber 200, a load lock chamber 300, an analysis device 500, and a plurality of pumps 610 and 630.

The first chamber 100 may perform a first heating process of a small sample SE2. The small sample SE2 may be smaller than a wafer sample SE1 described in more detail later. In an example embodiment, the small sample SE2 may be a silicon wafer slice having a surface terminated with H or TiH₂. In an example embodiment, the small sample SE2 may have various shapes. For example, unlike the samples illustrated in FIGS. 1, 2A, and 2B, some of a plurality of small samples SE2 in the first chamber 100 may have a rectangular parallelepiped shape, others of the plurality of small samples SE2 in the first chamber 100 may have a cylindrical shape, and the others of the plurality of small samples SE2 in the first chamber 100 may have a hemispherical shape.

The first chamber 100 may include a first chamber housing 111, an accommodation housing 113, a first support 120, a first heating device 130, a temperature measurement sensor 140, a first chamber gate 150, a transfer module 160, a sample container 170, and a sample tray 180.

The first chamber housing 111 may provide a first processing space 111S in which a first heating process for generating a first reactive gas by heating the small sample SE2 is performed. A lower surface of the first chamber housing 111 may be connected to a first pipe P1.

The accommodation housing 113 may provide an accommodation space 113S in which the small sample SE2 is accommodated. The accommodation housing 113 may include an accommodation gate (not illustrated) on one side wall. The small sample SE2 may be carried into or out of the accommodation space 113S in the accommodation housing 113 through the accommodation gate.

The first chamber housing 111 may communicate with the accommodation housing 113. A first chamber gate 150 may be between the first chamber housing 111 and the accommodation housing 113. In an example embodiment, a vertical length (a Z-direction length) of the first chamber housing 111 may be greater than a vertical length (the Z-direction length) of the accommodation housing 113. In an example embodiment, the first chamber housing 111 may have a rectangular parallelepiped shape, and the accommodation housing 113 may have a cylindrical shape.

The first support 120 may be in the first processing space 111S of the first chamber housing 111. The first support 120 may be configured to support the small sample SE2 and the sample container 170 storing the small sample SE2 while the first heating process is performed.

The first heating device 130 may be on a lower surface of the first support 120. The first heating device 130 may be configured to heat the sample container 170 storing the small sample SE2. When the first heating device 130 heats the sample container 170, heat may be transferred from the sample container 170 to the small sample SE2 to perform the first heating process. The first heating device 130 may be, for example, a halogen lamp that heats the sample container 170 by using a light source, but is not limited thereto.

The temperature measurement sensor 140 may be included in the first support 120 in the first processing space 111S of the first chamber housing 111. The temperature measurement sensor 140 may measure a temperature of the sample container 170 supported by the first support 120. The temperature measurement sensor 140 may be, for example, a contact-type temperature measurement sensor that measures a temperature of the sample container 170 by coming into contact with the sample container 170.

The first chamber gate 150 may be between the first chamber housing 111 and the accommodation housing 113. The first chamber gate 150 may open or close a space between the first chamber housing 111 and the accommodation housing 113 by moving in the vertical direction (the Z direction). For example, when the small sample SE2 and the sample container 170 storing the small sample SE2 are transferred from the accommodation space 113S to the first processing space 111S, the first chamber gate 150 may move downward in the vertical direction (the Z direction) to open a space between the first chamber housing 111 and the accommodation housing 113 by. On the other hand, when the first heating process is performed in the first processing space 111S, the first chamber gate 150 may move upward in the vertical direction (the Z direction) to close the space between the first chamber housing 111 and the accommodation housing 113.

The transfer module 160 may be in the accommodation space 113S of the accommodation housing 113. Specifically, the transfer module 160 may be on an upper surface of the accommodation housing 113 in the accommodation space 113S of the accommodation housing 113. The transfer module 160 may transfer the small sample SE2 and the sample container 170 storing the small sample SE2 from the accommodation space 113S to the first processing space 111S or may transfer the small sample SE2 on which the first heating process is completed and the sample container 170 storing the small sample SE2 from the first processing space 111S to the accommodation space 113S.

In an example embodiment, the transfer module 160 may include a fixed portion 161, a body portion 163, an arm portion 165, at least one first hand portion 167, and at least one second hand portion 169.

The fixed portion 161 may be connected to an upper surface of the accommodation housing 113. The transfer module 160 may be fixed to the accommodation housing 113 by the fixed portion 161.

The body portion 163 may be connected to the fixed portion 161. In an example embodiment, the body portion 163 may have a rectangular parallelepiped shape extending along a first direction D1, and a length of the body portion 163 may increase or decrease in the first direction D1. The arm portion 165 may be connected to the body portion 163. In an example embodiment, the arm portion 165 may have a rectangular parallelepiped shape extending in a vertical direction (the Z direction), and a length of the arm portion 165 may increase or decrease in the vertical direction (the Z direction). The transfer module 160 may reach the sample container 170 on the sample tray 180 by adjusting a length of the body portion 163 and a length of the arm portion 165.

The first hand portion 167 may be connected to the arm portion 165, and the second hand portion 169 may be connected to the first hand portion 167. The first hand portion 167 and the second hand portion 169 may hold the sample container 170. In an example embodiment, the second hand portion 169 may rotate. In detail, the second hand portion 169 may rotate on a Z-X plane to hold a container protrusion 173 of the sample container 170. In an example embodiment, the second hand portion 169 may have an L shape, and accordingly, the second hand portion 169 may hold the sample container 170 by engaging with the container protrusion 173 of the sample container 170 to be described below to transfer the sample container 170. However, a shape of the second hand portion 169 is not limited thereto and may have various shapes. In addition, the second hand portion 169 may transfer the sample container 170 in various ways regardless of a physical shape of the sample container 170. For example, the second hand portion 169 may also transfer the sample container 170 by holding the sample container 170 through vacuum suction, unlike the above description.

In an example embodiment, the first hand portion 167 may include a plurality of hand portions and the second hand portion 169 may include a plurality of hand portions. For example, there may be four first hand portions 167 and four second hand portions 169. In this case, the plurality of first hand portions 167 and the plurality of second hand portions 169 may be connected to one arm portion 165. In an example embodiment, each of the plurality of first hand portions 167 may be separated from each of the plurality of second hand portions 169 by the same distance. Specifically, as illustrated in FIG. 2B, the four first hand portions 167 may be separated from each other by the same distance in a vertical direction (the Z direction) on a vertical plane (the X-Y plane).

The sample container 170 may be in the accommodation space 113S. Specifically, the sample container 170 may be on the sample tray 180 in the accommodation space 113S. The sample container 170 may store the small sample SE2.

In an example embodiment, the sample container 170 may include a container body portion 171 having a cylindrical shape and a container protrusion 173 extending from the container body portion 171 in a horizontal directions (X and Y directions). Because the sample container 170 includes the container protrusion 173, the small sample SE2 and the sample container 170 storing the small sample SE2 may be transferred from the accommodation space 113S to the first processing space 111S by the transfer module 160.

In an example embodiment, a plurality of sample containers 170 may be on the sample tray 180. In this case, the plurality of sample containers 170 may be on the sample tray 180 to be separated from each other by the same distance. Specifically, as illustrated in FIG. 2B, in some embodiments, six sample containers 170 storing the small samples SE2 may be separated from each other by the same distance and may be on the sample tray 180. Although FIG. 2B illustrated that the six sample containers 170 are on the sample tray 180, embodiments are not limited thereto.

In an example embodiment, the sample container 170 may be made of a material with high thermal conductivity and high heat resistance. For example, the sample container 170 may be made of quartz or stainless. The sample container 170 is made of a material with high thermal conductivity and high heat resistance, and accordingly, when the sample container 170 is heated to perform the first heating process, heat may be well transferred from the sample container 170 to the small sample SE2 and the sample container 170 may not be affected by the first heating process.

The sample tray 180 may be in the accommodation space 113S. Specifically, the sample tray 180 may be on a lower surface of the accommodation housing 113 in the accommodation space 113S. The sample tray 180 may support the sample container 170. In an example embodiment, the sample tray 180 may have a donut shape. However, embodiments are not limited thereto and, in some embodiments, the sample tray 180 may have various shapes that may support the sample container 170 on an upper surface of the sample tray 180. For example, the sample tray 180 may have a circular shape, and the sample container 170 may be arranged on a circumference of the circular shape. In another example, the sample tray 170 may have a polygonal shape, and the sample container 170 may also be at an inner portion of an edge along the edge of the polygonal shape. In an example embodiment, the sample tray 180 may be configured to rotate. As the sample tray 180 rotates, the sample container 170 on the sample tray 180 and the small sample SE2 stored in the sample container 170 may be provided in a position where the sample container 170 and the small sample SE2 may be held by the transfer module 160.

The second chamber 200 may perform a second heating process of a wafer sample SE1. In an example embodiment, the wafer sample SE1 may include hydrogen (H). In an example embodiment, a size of the wafer sample SE1 may be larger than a size of the small sample SE2. For example, the wafer sample SE1 may have a diameter of about 100 mm to about 300 mm, and the small sample SE2 may have a diameter of about 10 mm to about 20 mm.

The second chamber 200 may include a second chamber housing 210, a second support 220, and a second heating device 230.

The second chamber housing 210 may include a second processing space 210S in which the second heating process for generating a second reactive gas by heating the wafer sample SE1 is performed.

The second support 220 may be in the second processing space 210S of the second chamber housing 210. The second support 220 may support the wafer sample SE1 while the second heating process is performed. The second support 220 may be, for example, an electrostatic chuck supporting the wafer sample SE1 by using electrostatic force but is not limited thereto.

The second heating device 230 may be under the second support 220 in the second processing space 210S of the second chamber housing 210. The second heating device 230 may heat the wafer sample SE1. The second heating device 230 may be, for example, a halogen lamp, but is not limited thereto.

A temperature measurement sensor (not illustrated) may be in the second support 120. The temperature measurement sensor may measure a temperature of the wafer sample SE1. The temperature measurement sensor may be, for example, a contact-type temperature measurement sensor.

The load lock chamber 300 may be connected to the second chamber 200. The wafer sample SE1 may be carried into or out of the second chamber 200 through the load lock chamber 300. A second chamber gate 400 may be between the load lock chamber 300 and the second chamber 200. When the second chamber gate 400 is opened or closed, the load lock chamber 300 may or may not communicate with the second chamber 200. For example, when the wafer sample SE1 is carried into the second chamber 200 through the load lock chamber 300, the second chamber gate 400 may be opened.

The analysis device 500 may analyze physical property of the small sample SE2 by using the first reactive gas generated from the small sample SE2 through the first heating process and analyze physical property of the wafer sample SE2 by using the first reactive gas generated from the wafer sample SE2 through the second heating process. In some embodiments, the analysis device 500 may be, for example, a mass spectrometer.

The first pipe P1 may be connected to the first chamber housing 111 of the first chamber 100. The first reactive gas generated by the first heating process may move from the first chamber housing 111 toward the analysis device 500 through the first pipe P1.

A first chamber valve V1 may be connected to the first pipe P1. Opening and closing of the first chamber valve V1 may be controlled by a control device 800. When the first chamber valve V1 is opened, the first reactive gas may move from the first chamber housing 111 toward the analysis device 500, and when the first chamber valve V1 is closed, the first reactive gas may not move from the first chamber housing 111 toward the analysis device 500.

A second pipe P2 may be connected to the second chamber housing 210 of the second chamber 200. The second reactive gas generated by the second heating process may move from the second chamber housing 210 toward the analysis device 500 through the second pipe P2.

A second chamber valve V2 may be connected to the second pipe P2. Opening and closing of the second chamber valve V2 may be controlled by the control device 800. When the second chamber valve V2 is opened, the second reactive gas may move from the second chamber housing 210 toward the analysis device 500, and when the second chamber valve V2 is closed, the second reactive gas may not move from the second chamber housing 210 toward the analysis device 500.

A third pipe P3 may be connected to the analysis device 500. The first reactive gas moved through the first pipe P1 or the second reactive gas moved through the second pipe P2 may move to the analysis device 500 through the third pipe P3.

An analysis device valve V3 may be connected to the third pipe P3. Opening and closing of the analysis device valve V3 may be controlled by the control device 800. When the analysis device valve V3 is opened, the first reactive gas moved through the first pipe P1 or the second reactive gas moved through the second pipe P2 may move to the analysis device 500, and when the analysis device valve V3 is closed, the first reactive gas moved through the first pipe P1 or the second reactive gas moved through the second pipe P2 may move to the analysis device 500.

A fourth pipe P4 may be connected to the plurality of pumps 610 and 630. The plurality of pumps 610 and 630 may maintain an internal state of the first chamber 100 and an internal state of the second chamber 200 in a vacuum state. The plurality of pumps 610 and 630 may be, for example, dry vacuum pumps or a turbo vacuum pumps.

A pump valve V4 may be connected to the fourth pipe P4. According to opening or closing of the pump valve V4, the internal state of the first chamber 100 and the internal state of the second chamber 200 may be adjusted.

A gas supply device 700 may be connected to the first chamber 100 and the second chamber 200. Specifically, the gas supply device 700 may be connected to the second chamber 200 through a fifth pipe P5 and connected to the first chamber 100 through a sixth pipe P6. The gas supply device 700 may supply a carrier gas to the first processing space 111S of the first chamber 100 and the second processing space 210S of the second chamber 200. Specifically, the gas supply device 700 may supply a carrier gas into the first chamber 100 after the first heating process is performed in the first chamber 100 and may supply a carrier gas into the second chamber 200 after the second heating process is performed in the second chamber 200. The carrier gas supplied to the first chamber 100 is mixed with the first reactive gas generated by the first heating process and move to the analysis device 500 through the first pipe P1 and the third pipe P3. In some embodiments, the carrier gas supplied to the second chamber 200 may be mixed with the second reactive gas generated by the second heating process and move to the analysis device 500 through the second pipe P2 and the third pipe P3. The carrier gas may be N₂ gas but embodiments are not limited thereto.

The control device 800 may control operations of the first chamber valve V1, the second chamber valve V2, and the analysis device valve V3. For example, the control device 800 may transmit and receive electrical signals to and from the first chamber valve V1, the second chamber valve V2, and the analysis device valve V3 to control the operations of the first chamber valve V1, the second chamber valve V2, and the analysis device valve V3.

The control device 800 may be implemented in hardware, firmware, software, or any combination thereof. For example, the control device 800 may be a computing device, such as a workstation computer, a desktop computer, a laptop computer, or a tablet computer. For example, the control device 800 may include a memory device, such as a read only memory (ROM) and a random access memory (RAM), and a processor configured to perform preset arithmetic operations and algorithms, such as a microprocessor, a central processing unit (CPU), or a graphics processing unit (GPU). In some embodiments, the control device 800 may include a receiver and a transmitter for receiving and transmitting electrical signals.

A related art sample analysis apparatus includes only a single chamber to analyze various types of samples by using only the single chamber. However, the single chamber is optimized only for a relatively large sample (for example, a silicon wafer having a diameter of about 100 mm to about 300 mm), and accordingly, the related art sample analysis apparatus has difficulty in analyzing a relatively small sample (for example, a silicon wafer slice having a diameter of about 10 mm to about 30 mm) by using the single chamber. In contrast, the sample analysis apparatus 10 according to various example embodiments includes the first chamber 100 for analyzing a relatively small sample and the second chamber 200 for analyzing a relatively large wafer sample. Accordingly, various types of samples may be analyzed in a more suitable chamber selected from the first chamber 100 and the second chamber 200, and thus, various types of samples may be analyzed quickly and efficiently, and consistency of analysis data may be increased.

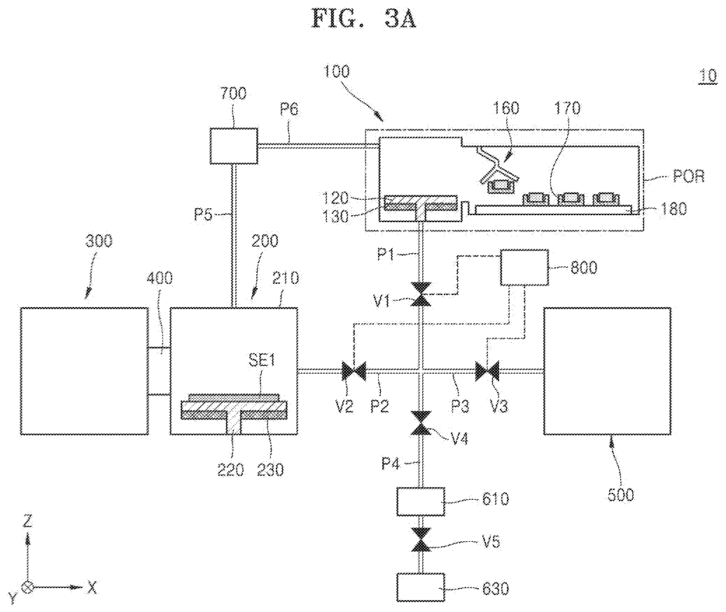

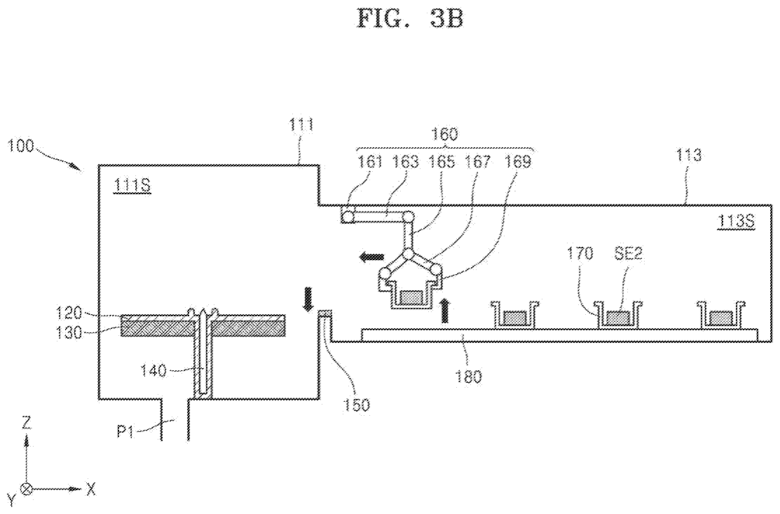

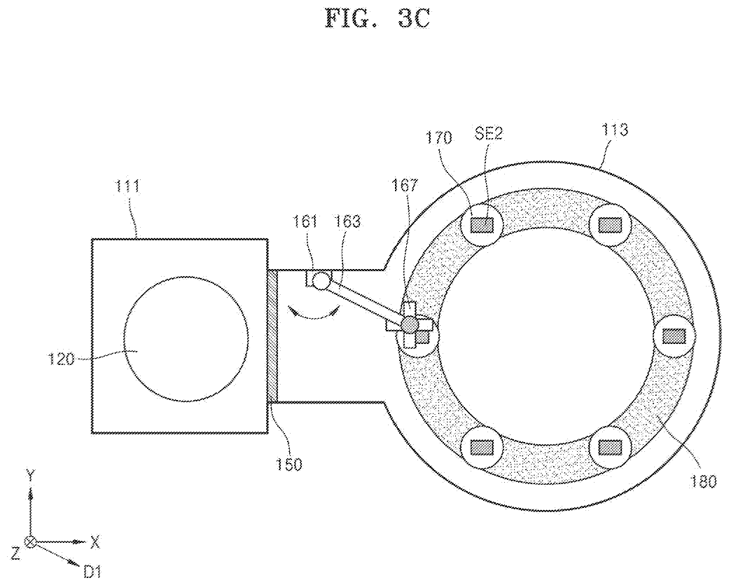

FIGS. 3A to 3C, FIGS. 4A to 4C, and FIGS. 5A to 5C, and 6 illustrate operating methods of the sample analysis apparatus 10. Specifically, FIGS. 3A, 4A, 5A, and 6 are configuration diagrams illustrating operating methods of the sample analysis apparatus 10, FIGS. 3B, 4B, and 5B are enlarged cross-sectional views illustrating the operating methods of the sample analysis apparatus 10, and FIGS. 3C, 4C, and 5C are plan views illustrating operating methods of the sample analysis apparatus 10.

Referring to FIGS. 3A, 3B, and 3C, first, the first chamber gate 150 and the analysis device valve V3 may be opened. Specifically, the first chamber gate 150 may be opened by moving downward in the vertical direction (the Z direction), and the analysis device valve V3 may be opened by the control device 800.

Next, the transfer module 160 may hold the small sample SE2 in the accommodation space 113S and the sample container 170 storing the small sample SE2. Specifically, a length of the arm portion 165 of the transfer module 160 is increased in the vertical direction (the Z direction), and then, the second hand portion 169 of the transfer module 160 may rotate in an inward direction (a direction toward the sample container 170) to hold the container protrusion 173 of the sample container 170. Next, the length of the arm portion 165 is reduced in the vertical direction (the Z direction), and accordingly, the sample container 170 may be lifted from the sample tray 180 in the vertical direction (the Z direction).

Next, the body portion 163 of the transfer module 160 may rotate around the fixed portion 161 on a plane (the X-Y plane) perpendicular to the vertical direction. Accordingly, the sample container 170 held by the second hand portion 169 may be transferred from the accommodation space 113S toward the first processing space 111S.

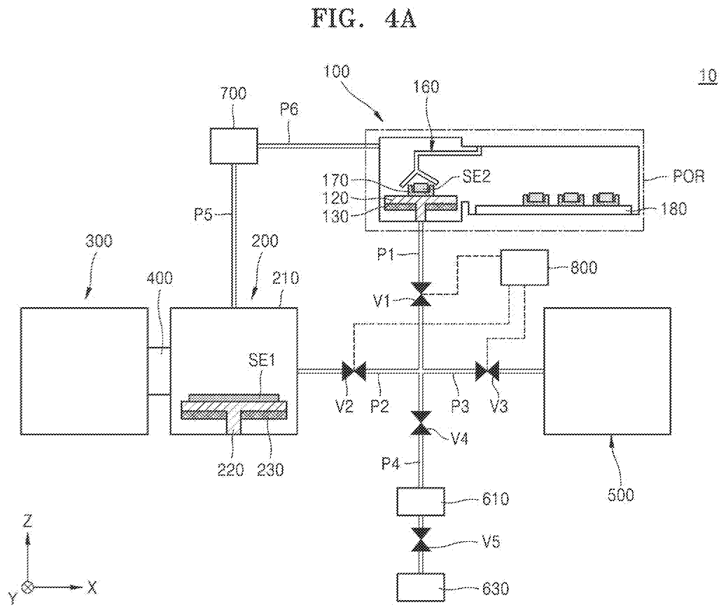

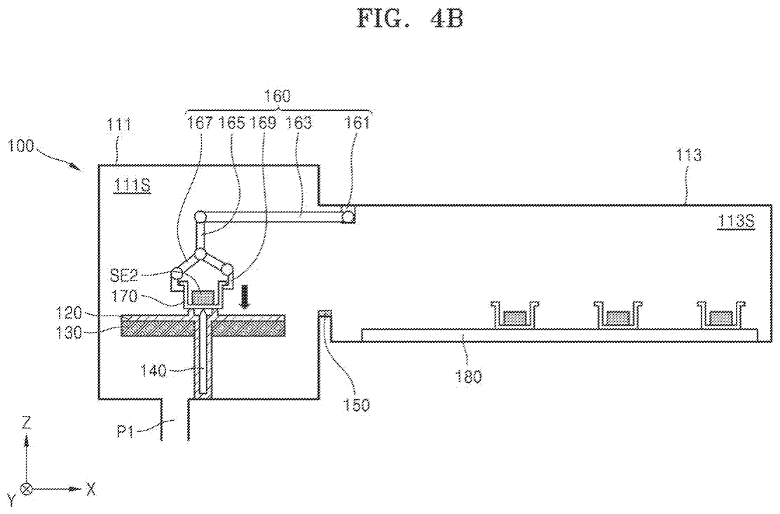

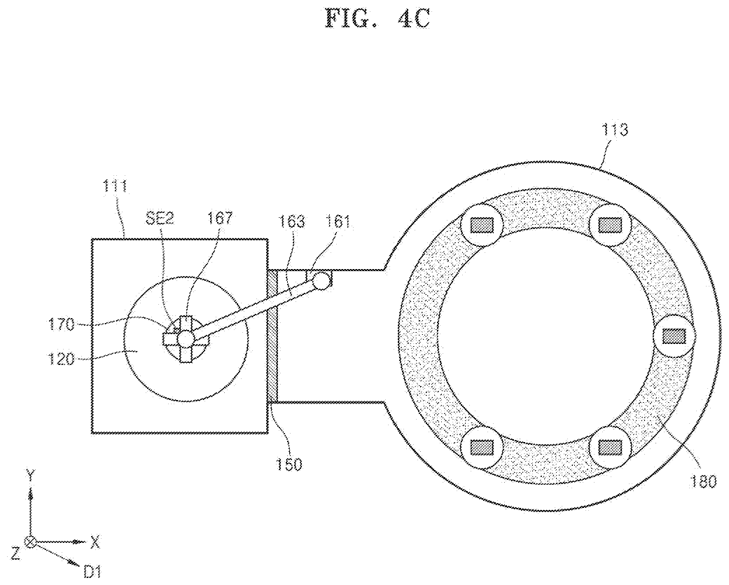

Referring to FIGS. 4A, 4B, and 4C, the small sample SE2 and the sample container 170 storing the small sample SE2 may be safely placed on the first support 120 as a result of operations of FIGS. 3A, 3B, and 3C. Specifically, the small sample SE2 and the sample container 170 storing the small sample SE2 may be aligned to overlap the first support 120 in the vertical direction by the rotation of the body portion 163 of the transfer module 160 described above. Next, as the length of the arm portion 165 of the transfer module 160 is increased in the vertical direction (the Z direction), the sample container 170 may be safely placed on the first support 120.

Next, the second hand portion 169 of the transfer module 160 may rotate in an outward direction (a direction away from the sample container 170), and accordingly, the second hand portion 169 does not hold the sample container 170.

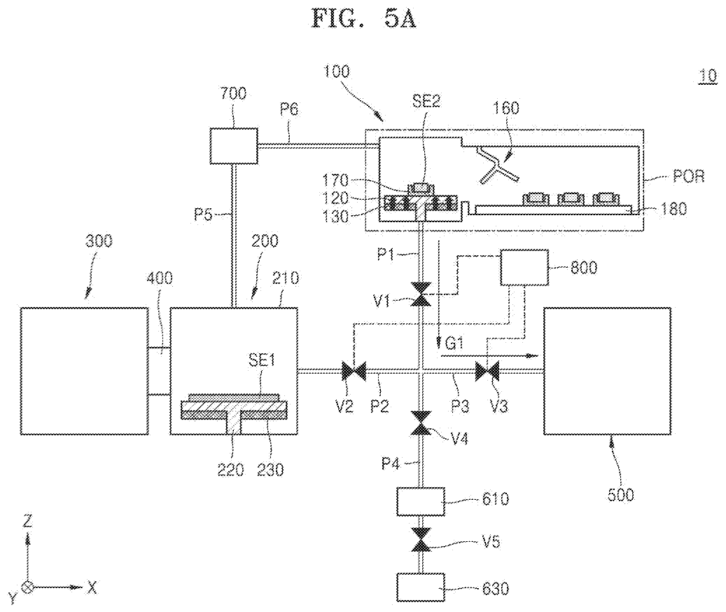

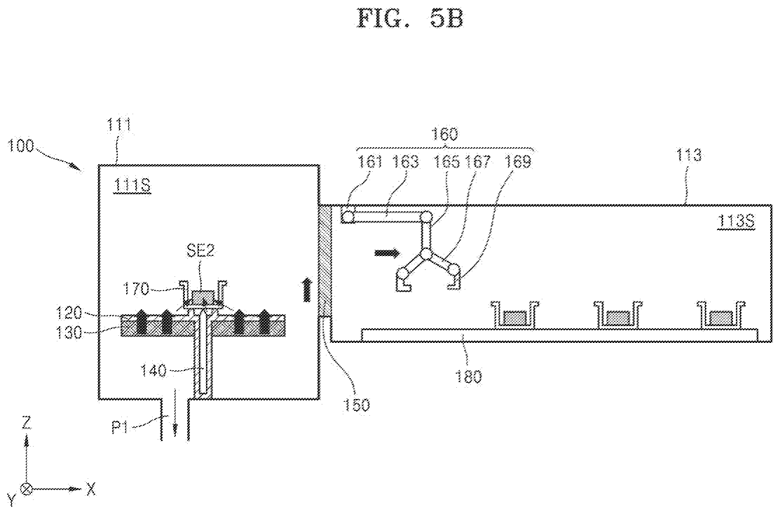

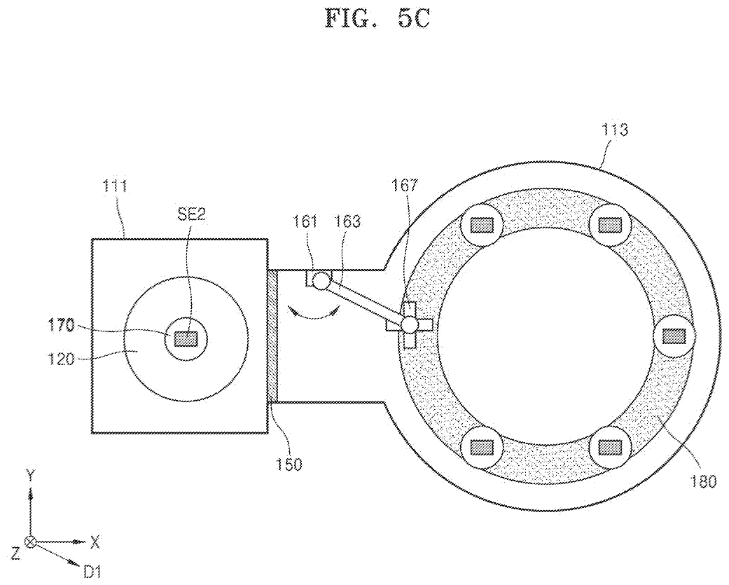

Referring to FIGS. 5A, 5B, and 5C, as a result of operations of FIGS. 4A, 4B, and 4C, the body portion 163 of the transfer module 160 may rotate toward the accommodation space 113S around the fixed portion 161 on a plane (the X-Y plane) perpendicular to the vertical direction to be out of the first processing space 111S. Next, the first chamber gate 150 may move upward in the vertical direction (the Z direction), and accordingly, the first chamber housing 111 of the first chamber 100 may not communicate with the accommodation housing 113 of the first chamber 100.

Next, a first heating process may be performed on the small sample SE2 in the first processing space 111S of the first chamber housing 111. Specifically, the first heating process may be performed by heating the sample container 170 by using the first heating device 130 and transferring heat from the heated sample container 170 to the small sample SE2. The temperature measurement sensor 140 may measure the temperature of the sample container 170 while the first heating process is performed. The first heating process may be adjusted according to the measured temperature. A first reactive gas G1 may be generated, on a surface of the small sample SE2, due to thermal desorption by the first heating process.

Next, the first chamber valve V1 may be opened by the control device 800. As the first chamber valve V1 is opened, the first reactive gas G1 may be supplied to the analysis device 500 through the first pipe P1 and the third pipe P3. The analysis device 500 may analyze physical property of the small sample SE2 by using the supplied first reactive gas G1. After the analysis of the physical property of the small sample SE2 is completed, the first chamber valve V1 and the analysis device valve V3 may be closed by the control device 800.

In an example embodiment, after the analysis of the small sample SE2 using the first reactive gas G1 is completed, the small sample SE2 subjected to the first heating process and the sample container 170 storing the small sample SE2 may be returned from the first processing space 111S to the accommodation space 113S by the transfer module 160. Specifically, after the first chamber valve V1 is closed, the first chamber gate 150 may be opened. Next, the transfer module 160 may move from the accommodation space 113S to the first processing space 111S and hold the sample container 170 storing the small sample SE2 subjected to the first heating process. Next, the transfer module 160 may move from the first processing space 111S to the accommodation space 113S while holding the sample container 170, and put down the sample container 170 onto the sample tray 180. Thereafter, the sample tray 180 may rotate and place the small sample SE2 subjected to the first heating process and the sample container 170 storing the small sample SE2 to be adjacent to an accommodation gate (not illustrated) included in one sidewall of the accommodation housing 113. The small sample SE2 and the sample container 170 storing the small sample SE2, which are adjacent to the accommodation gate, may be carried out of the accommodation housing 113 through the accommodation gate.

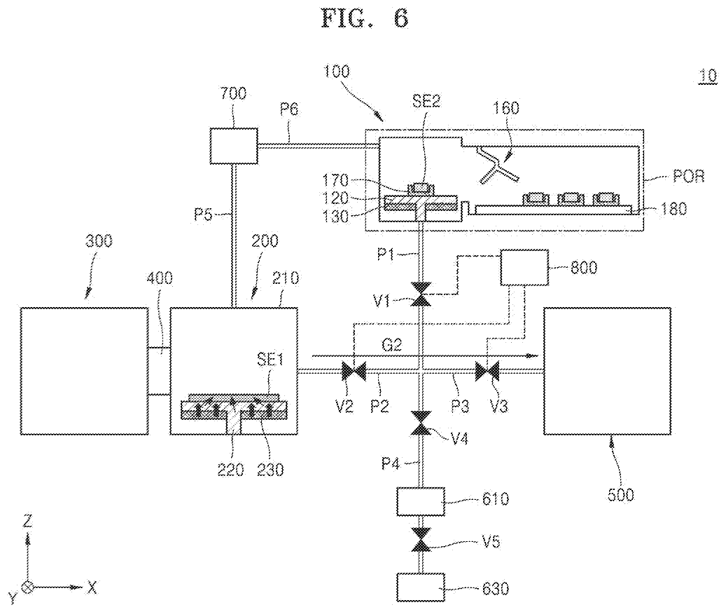

Referring to FIG. 6, as a result of FIGS. 5A, 5B, and 5C, a second heating process may be performed on the wafer sample SE1 in the second processing space 210S of the second chamber housing 210. The second reactive gas G2 may be generated, on the surface of the wafer sample SE1, due to thermal desorption by the second heating process. In this case, the wafer sample SE2 may also be carried into the second chamber 200 from the load lock chamber 300 after the analysis of the first reactive gas G1 is completed or may also be carried into the second chamber 200 from the load lock chamber 300 before the analysis of the first reactive gas G1 is completed.

Next, the second chamber valve V2 and the analysis device valve V3 may be opened by the control device 800. As the second chamber valve V2 and the analysis device valve V3 are opened, the second reactive gas G2 may be supplied to the analysis device 500 through the second pipe P2 and the third pipe P3. The analysis device 500 may analyze physical property of the wafer sample SE1 by using the supplied second reactive gas G2. After the analysis of the physical property of the wafer sample SE1 is completed, the second chamber valve V2 and the analysis device valve V3 may be closed by the control device 800.

As described above, example embodiments are disclosed in the drawings and specifications. The embodiments are described by using specific terms and are only used for the purpose of describing the technical idea of the present disclosure and are not used to limit the scope of the embodiments described in the meaning or claims. Therefore, those skilled in the art will understand that various modifications and equivalent other embodiments may be made therefrom. Therefore, the true technical scope of protection should be determined by the technical idea a recited in the appended claims.

While various embodiments have been particularly shown and described with reference to the drawings, it will be understood that various changes in form and details may be made therein without departing from the spirit and scope of the following claims.

What is claimed is:

1. A sample analysis apparatus comprising:
a first chamber configured to perform a first heating process for heating a small sample to generate a first reactive gas, the first chamber comprising:
a first chamber housing including a first processing space in which the first heating process is performed;
a first support configured to support the small sample while the first heating process is performed;
a first heating device arranged on a lower surface of the first support and configured to heat the small sample;
an accommodation housing including an accommodation space in which at least one small sample is accommodated, the accommodation housing being connected to the first chamber housing;
a sample tray arranged in the accommodation space;
at least one sample container arranged on the sample tray and configured to store the at least one small sample; and
a transfer module configured to transfer a sample container of the at least one sample container that stores the small sample from the accommodation space to the first processing space; and
a second chamber including a second chamber housing including a second processing space in which a second heating process for heating a wafer sample to generate a second reaction gas is performed, a second support configured to support the wafer sample while the second heating process is performed, and a second heating device arranged on a lower surface of the second support and configured to heat the wafer sample.

2. The sample analysis apparatus of claim 1, wherein a horizontal length of the first chamber housing is less than a horizontal length of the second chamber housing, and a vertical length of the first chamber housing is less than a vertical length of the second chamber housing.

3. The sample analysis apparatus of claim 1, wherein the transfer module comprises:
a fixed portion connected to the accommodation housing;
a body portion connected to the fixed portion and configured to be rotatable;
an arm portion connected to the body portion and configured to extend in a vertical direction; and
at least one hand portion connected to the arm portion and configured to hold the at least one sample container.

4. The sample analysis apparatus of claim 3, wherein the at least one hand portion includes four hand portions separated from each other by a same distance and connected to the arm portion.

5. The sample analysis apparatus of claim 1, wherein the at least one sample container comprises:
a container body; and
a container protrusion extending from the container body in a horizontal direction.

6. The sample analysis apparatus of claim 1, wherein the at least one sample container includes a plurality of sample containers that are separated from each other by a same distance on the sample tray.

7. The sample analysis apparatus of claim 1, wherein the sample tray is configured to be rotatable.

8. The sample analysis apparatus of claim 1, further comprising a first chamber gate configured to open or close the first chamber housing and the accommodation housing.

9. A sample analysis apparatus comprising:
a first chamber configured to perform a first heating process for heating a small sample to generate a first reactive gas, the first chamber comprising:
a first chamber housing including a first processing space in which the first heating process is performed;
a first support configured to support the small sample while the first heating process is performed;
a first heating device arranged on a lower surface of the first support and configured to heat the small sample;
an accommodation housing including an accommodation space in which at least one small sample is accommodated, the accommodation housing being connected to the first chamber housing;
a sample tray arranged in the accommodation space;
at least one sample container arranged on the sample tray and configured to respectively store the at least one small sample; and
a transfer module configured to transfer a sample container of the at least one sample container that stores the small sample from the accommodation space to the first processing space;
a second chamber including a second chamber housing including a second processing space in which a second heating process for heating a wafer sample to generate a second reactive gas is performed, a second support configured to support the wafer sample while the second heating process is performed, and a second heating device arranged on a lower surface of the second support and configured to heat the wafer sample;
an analysis device configured to analyze a physical property of the at least one small sample by using the first reactive gas and a physical property of the wafer sample by using the second reactive gas;
a first pipe configured to move the first reactive gas from the first chamber toward the analysis device;
a second pipe configured to move the second reactive gas from the second chamber toward the analysis device; and
a third pipe connected to the first pipe and the second pipe and configured to move the first reactive gas moving in the first pipe and the second reactive gas moving in the second pipe toward the analysis device.

10. The sample analysis apparatus of claim 9, further comprising:
a first chamber valve connected to the first pipe;
a second chamber valve connected to the second pipe; and
an analysis device valve connected to the third pipe,
wherein the first chamber valve and the second chamber valve are alternately opened or closed.

11. The sample analysis apparatus of claim 10, further comprising a control device configured to control opening or closing of the first chamber valve, the second chamber valve, and the analysis device valve.

12. The sample analysis apparatus of claim 9, further comprising a gas supply device configured to supply a carrier gas to the first processing space and the second processing space.

13. The sample analysis apparatus of claim 9, wherein a horizontal length of the first chamber housing is less than a horizontal length of the second chamber housing, and a vertical length of the first chamber housing is less than a vertical length of the second chamber housing.

14. The sample analysis apparatus of claim 9, wherein the transfer module comprises:
a fixed portion connected to the accommodation housing;
a body portion connected to the fixed portion and configured to be rotatable;
an arm portion connected to the body portion and configured to extend in a vertical direction; and
at least one hand portion connected to the arm portion and configured to hold the at least one sample container.

15. The sample analysis apparatus of claim 9, wherein the at least one small sample container includes one of quartz and stainless steel (SUS) and includes a container body and a container protrusion extending from the container body in a horizontal direction.

16. The sample analysis apparatus of claim 9, wherein the sample tray has a donut shape and is configured to be rotatable.

17. The sample analysis apparatus of claim 9, wherein the at least one sample container includes a plurality of sample containers that are separated from each other by a same distance on the sample tray.

18. The sample analysis apparatus of claim 9, wherein the analysis device is a mass spectrometer.

19. A sample analysis method comprising:
opening a first chamber gate and an analysis device valve;
transferring a sample container accommodated in an accommodation space in an accommodation housing and a small sample stored in the sample container into a first processing space in a first chamber housing by using a transfer module;
performing a first heating process on the small sample;
opening a first chamber valve to move a first reactive gas generated by the first heating process to an analysis device;
closing the first chamber valve and the analysis device valve after analysis of the first reactive gas is completed;
providing a wafer sample to a second processing space in a second chamber;
performing a second heating process on the wafer sample;
sequentially opening the analysis device valve and a second chamber valve to move a second reactive gas generated by the second heating process to the analysis device; and
closing the second chamber valve and the analysis device valve after analysis of the second reactive gas is completed.

20. The sample analysis method of claim 19, further comprising returning the small sample subjected to the first heating process and the sample container storing the small sample from the first processing space to the accommodation space after analysis of the first reactive gas is completed.
